(19) United States
(12) Plant Patent Application Publication
(10) Pub. No.: US 2024/0334851 P1
(43) Pub. Date: Oct. 3, 2024

(54) APPLE TREE NAMED MAIA-MITCHELL

(50) ;

(71) Applicant: Midwest Apple Improvement Association, Oberlin, OH (US)

(72) Inventor: William A Pitts, Sterling, NY (US)

(21) Appl. No.: 18/130,019

(22) Filed: Apr. 3, 2023

(51) Int. Cl. A01H 6/74; A01H 5/08

(52) U.S. Cl. CPC A01H 6/7418; A01H 5/08
USPC PLT/161

(57) ABSTRACT

A new and distinct variety of apple was identified as a limb mutation of the variety ‘MAIA1.’ The new variety, named ‘MAIA-MITCHELL’ ripens in late October. ‘MAIA-MITCHELL’ has a strong red coloration that is distinct from its parent in both hue and coverage. ‘MAIA-MITCHELL’ develops this coloration several weeks earlier than its parent variety, allowing growers to harvest the fruit at optimal maturity without the need to wait for commercially preferable coloration to develop.

Latin name:

Latin name of the genus and species of the plant claimed: Malus x domestica.

Variety denomination:

Variety denomination: ‘MAIA-MITCHELL’.

### BACKGROUND OF THE INVENTION

A new and distinct variety of Evercrisp™ apple was identified as a limb mutation of the Malus domestica variety ‘MAIA1’ (U.S. Plant Pat No. 24579). The limb mutation was initially identified in 2018 among trees growing on a commercial apple orchard in Wolcott, New York, located in Wayne County. The ‘MAIA1’ tree containing the mutation was planted in a commercial block on Budagovsky-9 (Bud-9 or B9, unpatented) rootstock in 2014. The novel variety described herein is identifiably distinct from the parent variety by the earlier, much brighter, and more complete red coloring of the fruit. Budwood from the original tree was collected and the new variety of apple tree was asexually reproduced by grafting on a Standard rootstock in Sterling, New York, located in Cayuga County. The first generation was grafted in 2019. Asexual propagation shows the unique combination of characteristics and distinct color are true to the description contained herein through successive generations. A third generation was budded in 2021 on three commercial rootstocks (Budagovsky-9, Geneva-11 and Geneva-935), also in Sterling New York. While chip-budding was used initially to reproduce the new apple tree variety, it is expected that other industry recognized grafting methods would be effective.

As a limb mutation, the parent ‘MAIA1’ is the most closely related commercial variety to the new apple variety described herein. The fruit of the apple tree of the new variety possesses an overall strong red color, distinct from the light orange-red of the parent variety. This color is present over approximately 90% of the fruit surface and develops up to eight weeks earlier than its parent. Other than the color, the fruit of the new variety exhibits the same shape, size, flavor, and texture of the parent variety.

The new variety, named ‘MAIA-MITCHELL’ is distinct from ‘MAIA1’ as ‘MAIA-MITCHELL’ possesses a much higher percentage of red coloration (up to 90%), a darker coloration (Strong-red 46A as opposed to Orange-Red N 34 A of the parent variety) , and shows significant fruit color up to eight weeks earlier than the parent variety. The fruit of ‘MAIA-MITCHELL’ are medium sized, extremely crisp, sweet, and with long storability. The development of early, significant coloration of apple fruits is a desirable commercial attribute, as it allows growers to harvest at optimum maturity without waiting for commercially desirable color to develop.

### BRIEF DESCRIPTION OF THE DRAWINGS

The accompanying photographs show typical specimens of the new variety as depicted in color as nearly true as is reasonably possible. ‘MAIA-MITCHELL’ photographs were taken in Wayne and Cayuga Counties, New York.

FIG. 1. Shows the original ‘MAIA-MITCHELL’ limb mutation (in foreground) on the parent ‘MAIA1’ tree, in Wolcott, New York. The photograph was taken Sep. 28, 2019 when the tree was five years old.

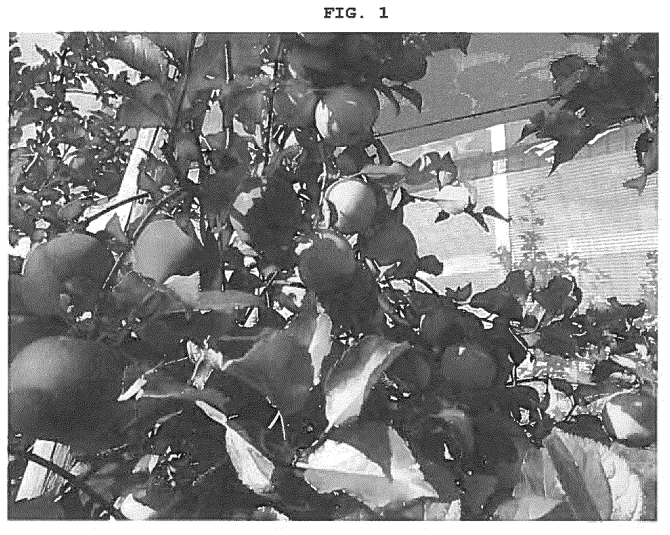

FIG. 2. Shows the difference in coloration between ‘MAIA1’ (Panel A) and ‘MAIA-MITCHELL’ (Panel B) on Aug. 15, 2022, roughly ten weeks prior to harvest. Both trees were grafted by chip-budding onto seedling rootstocks in 2020 in Sterling, New York.

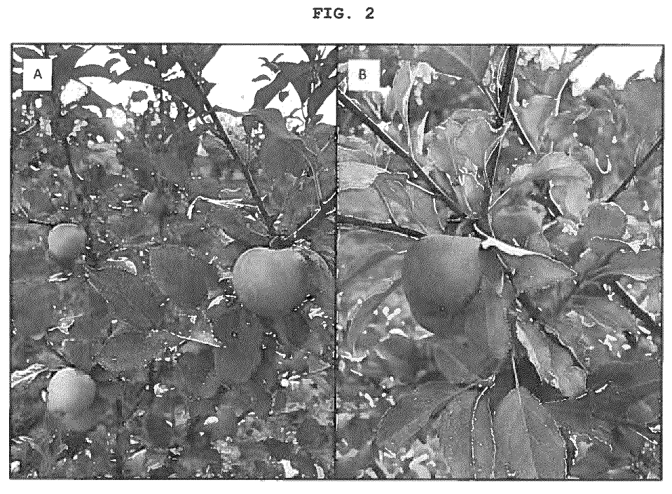

FIG. 3 Shows the difference in coloration between FIG. 3. ‘MAIA1’ (Panel A) and ‘MAIA-MITCHELL’ (Panel B) on Sep. 8, 2022, roughly seven weeks prior to harvest. Both trees were grafted by chip-budding onto seedling rootstocks in 2020 in Sterling, New York.

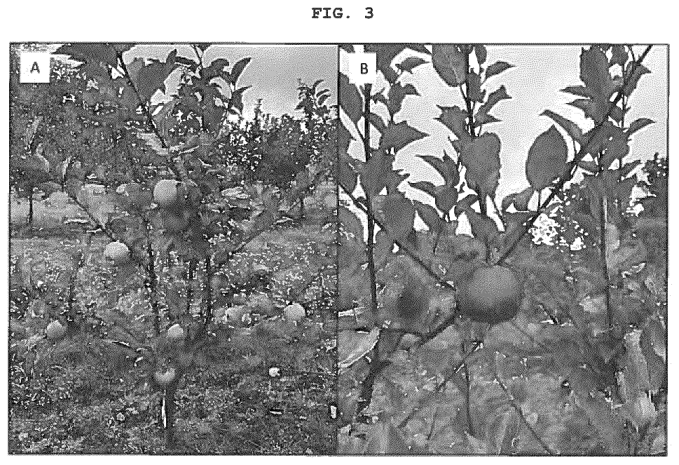

FIG. 4. Shows the difference in coloration between the mature fruit of ‘MAIA1’ (Right) and ‘MAIA-MITCHELL’ (Left) at the time of harvest.

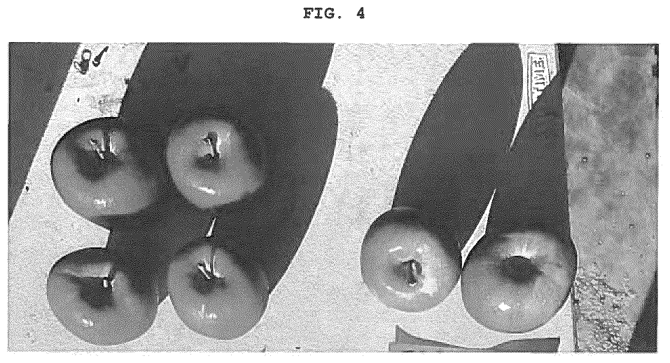

FIG. 5. Shows the iodine solution reactions for starch scoring of fruit of ‘MAIA1’ (Left) and ‘MAIA-MITCHELL’ (Right) harvested on the same day, illustrating comparable maturity timing.

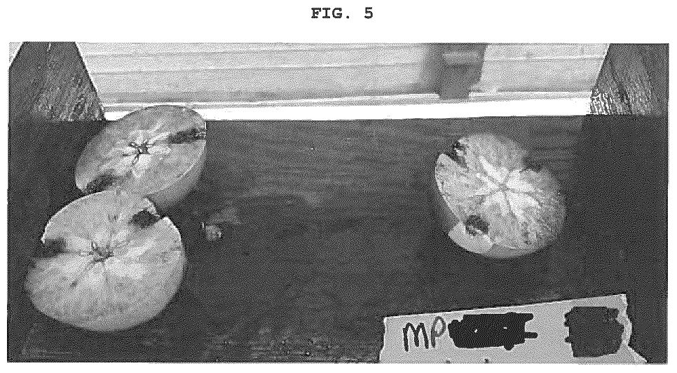

### DETAILED BOTANICAL DESCRIPTION

Color references are made to The Royal Horticultural Society Colour Chart (R.H.S.) 2001 Edition.
Parentage: Limb mutation of ‘MAIA1’ (U.S. Plant Pat No. 24579) . Locality of the original discovery and observations are Wolcott, New York and Sterling, New York.
Grafted tree on B9 rootstock (reference tree):
   Age.—5 years.
   Vigor.—Moderate.
   Form.—Round, spreading with moderate density
   Production.—Very productive; 2.5 bushels/tree at 300 trees/acre=750 bushels/acre.
   Growth type.—Non-spur with very weak dominance.
   Bearing.—Annual.
Trunk:
   Bark color.—Grey-Brown 199A.
   Lenticels.—Oblong, 2.5×1.5 mm.
   Lenticel color.—Grey Brown 199B.
   Lenticel density.—6 lenticels/cm² on average.
Branches:
   3 Year old.—16-22 mm in diameter, branch angle 80-90°, Grey-Brown 199A.
   2 Year old.—14-18 mm in diameter, branch angle 80° 90°, Grey-Brown 199A.
   1 Year old.—9-10 mm in diameter, branch angle 80° 90°, Grey-Brown 199D.
Leaves:
   Size.—Length 75 mm; width 55 mm.
   Texture.—Leathery, crisp.
   Form.—Ovate.
   Base.—Roundly cuneate.
   Apex.—Acute.
   Adaxial surface pubescence.—None.
   Abaxial surface pubescence.—Finely pubescent.
   Adaxial surface color.—Yellow-Green 146B.
   Abaxial surface color.—Yellow-Green 146D.
   Venation.—Pinnate, 8-9 major veins; mainly alternate; Yellow-Green 149D.
   Margin.—Serrate.
   Petiole length.—30 mm.
   Petiole width.—3 mm.
   Petiole abaxial color.—Yellow-Green 145A to Greyed Purple 185C toward base and abscission layer on larger leaves.
   Petiole groove.—Very shallow, depth 0.1 mm.
   Stipules.—Present; 5 mm length; 1 mm width; Yellow Green 146D.
   Leaf glands.—None observed.
Leaf buds:
   Length.—1.5 mm.
   Width.—1.5 mm.
   Color.—Yellow-Green 145B with Greyed-Purple 185B overtones.
   Placement on branch.—Alternate.
   Internode distance.—25 mm.
Spurs: Present on 2 yr and older wood.
   Length.—Varies from 10 to 350 mm.
   Proportion.—350 mm: 1; 200-350 mm: 3; 100-200 mm: 3; 50-100 mm: 3; 10-50 mm: 3.
   Width.—Short: 3.5 mm; Medium: 5 mm; Long: 7 mm.
Flowers at popcorn stage:
   Pedicel.—Length 12-15 mm; diameter 2 mm.
   Pedicel color.—Yellow-Green 148D.
   Bud.—Length 7-9 mm; width 7 mm.
   Bud color.—Red-Purple 63B, before flower petals expand and open; background of White 155C.
Flowers at full bloom:
   Bloom time.—Blooms with ‘Golden Delicious’ and ‘MAIA1.’ Bloom time varies with temperature.
   Bloom period.—Approximately 7 days but varies with temperature.
   Presentation.—Showy.
   Fragrance.—Aromatic.
   Fertility.—Fertile.
   Corolla diameter.—−35-40 mm.
   Number of flowers per cluster.—4-6 with 5 typical
Petals:
   Arrangement.—Bases overlapping; 5 petals per flower; each petal 15-18 mm length; 12-14 mm width.
   Color.—Upper surface (inside) White 155C; lower surface (outside) White 155C with Red-Purple 70B overtones pronounced as flower begins to open and then fading as flower completely opens.
   Shape.—Broadly ovate, abruptly cuneate at junction with receptacle.
   Veins.—Non-distinct.
   Margins.—Somewhat ruffled with occasional notching at the apex.
   Texture.—Soft.
   Receptacle.—Length 8 mm; width 6 mm; color Green 143D.
   Pedicel.—Length 18-22 mm; width 2 mm; color Green 143D when pubescent; Reddish tones 58A as pubescence is lost as pedicel matures.
   Sepals.—5/flower; wedge shaped; sharply pointed; length 6 mm; width 3 mm at widest point (attachment); color Greyed-Green 193B with Reddish Purple tip 58A.
   Stamens.—18-22 In number.
   Anthers.—Length 2 mm; width 1.5 mm; color Yellow 3D.
   Pollen.—Present.
   Filaments.—Length 5-12 mm, increasing in length as flower matures; width 0.5 mm; color Greyed-Green 193B
   Pistil.—Held lower than anthers in majority of blossoms,
   Ovary.—Length 4 mm; width 3 mm; color Green 139B in cross section with Reddish Purple 60A layer under exterior pubescence.
   Style.—Length 10 mm from fused base to Stigmas; width 1.5 mm at fused base; width 0.4 mm as separated styles; pubescent at point of separation from fused base; color Green 143A.
   Stigma.—Width 1 mm; color Green 143A.
Pollination requirements: Requires cross-pollination from diploid varieties with overlapping bloom.
Fruit:
   Maturity when described.—Freshly harvested.
   Date of picking.—Oct. 26, 2022, in Wayne County NY.
   Size.—Axial diameter 70-72 mm; Transverse diameter 80-85 mm.
   Fruit weight.—average 238 g.
   Form.—Roundish oblate regular.
   Cavity.—Acuminate medium deep; russet extending out of cavity.
   Basin.—Shape, depth, width, medium depth, medium width, Symetrical obtuse regular wavy.
   Calyx.—Closed reflexed.
Skin:
   Thickness.—Medium.
   Tendency to crack.—Little.
   Stripes.—None.
   Lenticels.—Round shape, 0.02-0.08 mm diameter.
   Color.—Strong-red, 46A.
   Ground color.—Typically Greyed-red 180A, when fully colored but ground color can range in small areas from Brownish-orange 172C to Greyed-red 179B.
Flesh:
   Aroma.—Sweet, aromatic.
   Color.—Yellow-white 159B.
   Texture.—Firm, crisp, juicy, and sweet.
   Eating quality.—Excellent.
   Flavor.—Indistinguishable from ‘MAIA1’.
Core: Medium size.
   Bundle area.—250 mm² .
   Bundle.—Inconspicuous.
   Seed cells.—Walls thin, tough.
Seeds:
   Number perfect.—10.
   Number in one cell.—2.
   Length.—9-10 mm.
   Breadth.—5 mm.
   Color.—Greyed-orange 165A.
Stem:
   Length.—23-24 mm.
   Width.—2.2 mm.
   Color.—Greyed-yellow 162A.
Use: Fresh market, dessert.
Shipping quality: Good.
Keeping quality: Excellent.
Drought tolerance: Average for domestic apple.
Tree Winter hardiness: Average for domestic apple.
Disease:
   Resistance.—No known resistance to pathogens.

What is claimed is:

1. A new, distinct apple tree variety named ‘MAIA-MITCHELL’, as illustrated and described herein.
